(19) United States
(12) Patent Application Publication
(10) Pub. No.: US 2018/0117000 A1
(43) Pub. Date: May 3, 2018

(54) ENERGY METABOLIC ACTIVATING AGENT FOR MUSCLE CELLS

(71) Applicant: ORYZA OIL & FAT CHEMICAL CO., LTD., Aichi (JP)

(72) Inventors: Kazuya TODA, Aichi (JP); Shogo TAKEDA, Aichi (JP); Hiroshi SHIMODA, Aichi (JP); Hiromichi MURAI, Aichi (JP)

(21) Appl. No.: 15/565,442

(22) PCT Filed: Mar. 24, 2016
(86) PCT No.: PCT/JP2016/059492

§ 371 Date: Oct. 10, 2017

(30) Foreign Application Priority Data
Apr. 10, 2015 (JP) 2015-081005

(51) Int. Cl. A61K 31/353; A61K 36/906; A61P 3/00; A23L 29/00

(52) U.S. Cl. CPC A61K 31/353; A23L 29/035; A61P 3/00; A61K 36/906

(57) ABSTRACT

This invention provides a new energy-metabolic activating agent for muscle cells and provides compounds that are widely used in foods and drinks and in medicines and in quasi-drugs or the like. The technical features of this invention are as follows:

(1) A sugar transporter (GLUT4) gene-expression promoting agent, including at least one active substance selected from the following: 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone,
(2) A sugar transporter (GLUT4) gene-expression promoting agent, including at least one active substance selected from either techtochrysin or 5,7-dimethoxyflavone, and
(3) A sugar transporter (GLUT4) gene-expression promoting agent within the muscle cells that includes any one of the chemical compounds shown in the following chemical formula 1. (Of such formula 1, R1 and R2, respectively, mean an alkyl group with hydrogen or with 1˜3-carbon.)

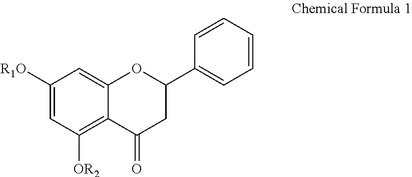

### TECHNICAL FIELD

This invention relates to a new energy-metabolic activating agent for muscle cells. This invention is widely used in foods and drinks and in medicines and in quasi-drugs or the like.

### TECHNICAL BACKGROUND

During exercise, much energy is spent within muscle cells, and it is known that a vast energy source is sugars (i.e. glucose or the like). Thus, the taking of sugar into the muscles is important in producing energy, which means that an increased ability to exercise can be expected upon taking more sugar into the muscles (see Patent Document 1). In this case, it is the sugar transporter GLUT4: glucose-transporter 4 that is involved in the process of sugar being taken into the muscles.

It is known that the factor PGC-1α relates to the energy-metabolic control of skeletal muscles (see Non-patent Document 2).

PGC-1α means Peroxisome Proliferator-activated Receptor γ Co-activator 1α and is known for promoting mitochondrial synthesis and in increasing the amount of GLUT4 that is the sugar-transporter in the taking of glucose (blood-sugar) into blood flowing into skeletal muscles. It is also known that the PGC-1α is a therapeutic target in the treating of life-style related diseases such as the metabolic syndrome that is induced by less PGC-1α being expressed, thus causing deceased mitochondrial-function due to diabetes or aging or to decreased energy consumption.

In exposing a mouse to a cooling environment, the amount of PGC-1α in the skeletal muscles of such a mouse increases, which shows that the PGC-1α relates to the control of heat-production in skeletal-muscle tissue. Forcibly expressing the PGC-1α induces an NRF that promotes transferring the factor relating to the mitochondrial-respiratory chain as well as to the expression of the uncoupling protein (UCP) that is considered necessary in promoting energy consumption in mitochondria and in inducing the expression of the mitochondrial-transcription factor A (mtTFA), which is important in replicating the mitochondrial genome and in processing the transcription reaction. Then, these molecular functions, in being expressed, thus increase the number of mitochondria within the muscle cells that now obviously show an increase in oxygen consumption within such cells.

Therefore, it is known that once the mitochondrial function in human cells is activated, the production of heat or the consumption of energy is induced, thus activating the metabolism of sugars and lipids that are the sources of energy within the muscle cells (see Non-patent Document 3).

So far, as motor-function improving agents, vitamins (Patent Document 1) and imidazole compounds (Patent Document 2) and ornithine (Patent Document 3), which are liberally contained in bonito fish and tuna fish, are known for being anti-fatigue agents.

### PRIOR ARTS

### Patent Documents

Patent Document 1: Japanese Published Unexamined Application No. 2010-138170
Patent Document 2: Japanese Published Unexamined Application No. 2002-338473
Patent Document 3: International Publication No. 2007/142286
Patent Document 4: Japanese Published Unexamined Application No. 2015-10078

### Non-Patent Documents

Non-patent Document 1: Hideo HATTA “Sports Training Methods Using Energy Metabolism,” 2004, by Kodansha, Ltd.
Non-patent Document 2: Cells, 92, 829-838, 1998 Non-patent Document 3:Cells. 98, 1165-124, 1999

### SUMMARY OF THE INVENTION

### Problems to be Resolved by the Invention

Because of this situation, the inventors promoted the expression of the sugar-transporter (GLUT4) gene within muscle cells regarding specified compounds contained in black ginger, and they activated the gene of the PGC-1α and found that the production of mitochondria DNA had increased.

Thus, they achieved this invention. In other words, this invention is intended to provide the new promoting agent for expressing the sugar transporter (GLUT4) gene within muscle cells; to provide the PGC-1α gene-activating agent; and to provide the energy metabolic-activating agent within muscle cells, thus producing muscle cells of an excellent quality. As an art associated with this invention, Patent Document 4 shows that black-ginger extract and polymethoxyflavone work in increasing muscle mass. However, this invention shows that such black-ginger extract and polymethoxyflavone work in enhancing the metabolic capability of muscle cells. Thus, this invention is clearly distinct from the invention of Patent Document 4 that focuses on increasing muscle mass. In other words, the increase in muscle mass is controlled by another process other than by the increase in the metabolic capability of the muscle. To increase muscle mass, it is important to increase muscle synthesis and to decrease muscle decomposition. Moreover, to improve the metabolic capability of muscle, it is also important to increase the intake-amount of nutrition (sugar, or the like) and the accumulation of glycogen and the amount of mitochondria in the muscle cells. In fact, to increase muscle mass, soybean-derived protein or milk-serum protein (whey protein) or the like is widely used. To improve the metabolic capability of muscle, carnitine or coenzyme Q10 or the like is commonly used. That is, these products are obviously used according to their intended purpose. Patent Document 4 shows an experiment on mice and an effect that was limited only to the soleus muscle and not to the other muscles being exercised. The inventors of this invention evaluated such effect by using the adjusted data regarding the following test examples and by verifying the fact that the metabolism of each muscle cell and not of the overall muscle cells is improved. Thus, they achieved this invention.

### Means for Resolving the Problems

To resolve the problems mentioned above, this invention has the following technical features.

(1) A sugar transporter (GLUT4) gene-expression promoting agent, including at least one active substance selected from the following: 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone.
(2) A sugar transporter (GLUT4) gene-expression promoting agent, including at least one active substance selected from either techtochrysin or 5,7-dimethoxyflavone.
(3) A sugar-transporter (GLUT4) gene-expression promoting agent within the muscle cells that includes any one of the chemical compounds as shown in the following Chemical Formula 1. (Of such Chemical Formula 1, R1 and R2, respectively, mean an alkyl group with hydrogen or with 1′ 3-carbon.)

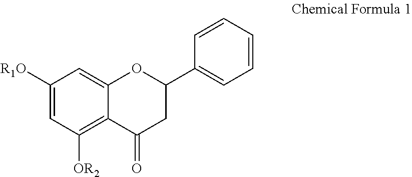

(4) A PGC-1α gene-expression promoting agent, including at least one active substance selected from the following: 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone.
(5) A PGC-1α gene-expression promoting agent, including at least one active substance selected from either techtochrysin or 5,7-dimethoxyflavone.
(6) A PGC-1α gene-expression promoting agent within the muscle cells, including any one of the chemical compounds as shown in the following Chemical Formula 1. (Of such Chemical Formula 1, R1 and R2 respectively mean an alkyl group with hydrogen or with 1˜3-carbon.)

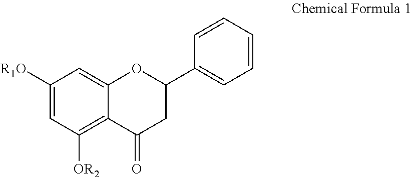

(7) An energy-metabolic activating agent of the muscle cells, including the substance described in any one of Claims 1 to 6.
(8) A sugar-transporter (GLUT4) gene-expression promoting agent, including black-ginger extract as an active substance.
(9) A PGC-1α gene-expression promoting agent, including black-ginger extract as an active substance.
(10) A composition of food for activating energy metabolism in the muscle cells, including techtochrysin as an active substance.
(11) A composition of food for activating energy metabolism in the muscle cells, including 5,7-dimethoxyflavone as an active substance.
(12) A method for activating energy metabolism in the muscle cells by administering to human beings at least one active substance selected from the following: 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone.

### BRIEF DESCRIPTION OF THE DRAWINGS

FIG. 1 is an isolated scheme of 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone.

FIG. 2 is a graph showing how the black-ginger extract (KPE) and the fractional separation (of Compounds 1-8) effect the mRNA expression of the sugar transporter (GLUT4).

FIG. 3 is a graph showing how the black-ginger extract (KPE) and the fractional separation (of Compounds 1-8) effect the mRNA expression of the PGC-1α.

### BEST MODE FOR CARRYING OUT THE INVENTION

Hereinafter is a detailed description of the invention.

The energy-metabolic activating agent of the muscle cells of this invention is characterized in including at least one compound selected from the following: 5-Hydroxy-3, 7-dimethoxyflavone; techtochrysin; 3,7,4′-trimethylkaempferol; retusine; pentamethylquercetin; trimethylapigenin; tetramethylkaempferol; and 5,7-dimethoxyflavone. (Hereinafter, these compounds shall simply be referred to as the “compound-group.”) The above referenced compound-group should be shown as the Chemical Formula 2, below.

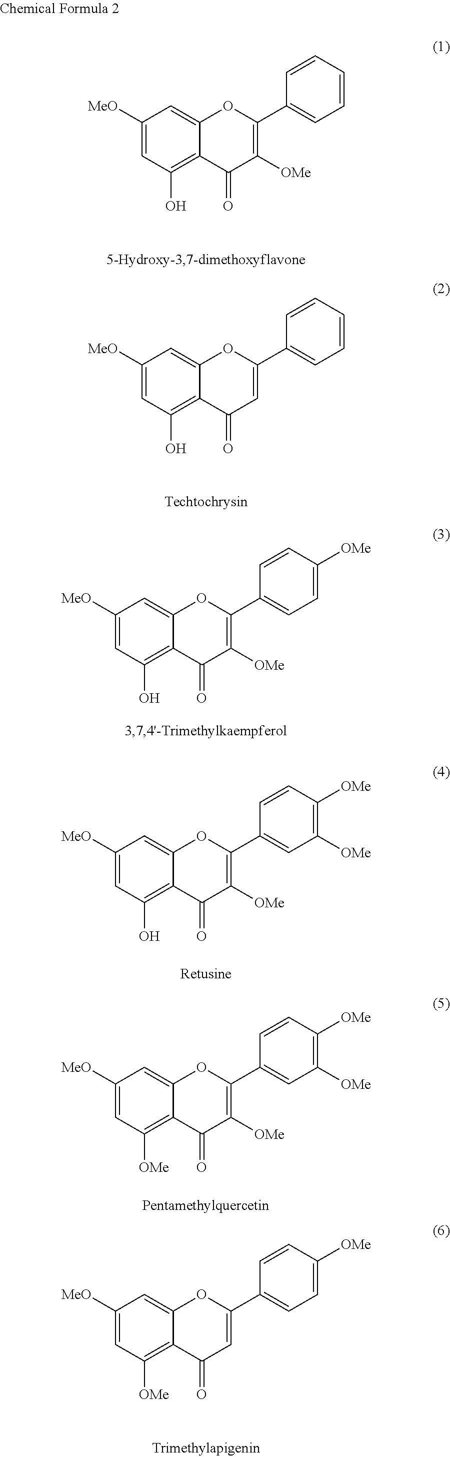 [SMILES: COc1cc(O)c2c(=O)c(OC)c(-c3ccccc3)oc2c1 ; COc1cc(O)c2c(=O)cc(-c3ccccc3)oc2c1 ; COc1ccc(-c2oc3cc(OC)cc(O)c3c(=O)c2OC)cc1 ; COc1cc(O)c2c(=O)c(OC)c(-c3ccc(OC)c(OC)c3)oc2c1 ; COc1cc(OC)c2c(=O)c(OC)c(-c3ccc(OC)c(OC)c3)oc2c1 ; COc1ccc(-c2cc(=O)c3c(OC)cc(OC)cc3o2)cc1]

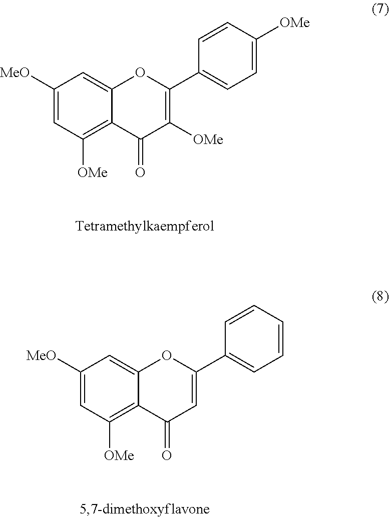 [SMILES: COc1ccc(-c2oc3cc(OC)cc(OC)c3c(=O)c2OC)cc1 ; COc1cc(OC)c2c(=O)cc(-c3ccccc3)oc2c1]

Of such compound-group, techtochrysin and 5,7-dimethoxyflavone are preferred.

The method used in obtaining the aforementioned compound-group is not limited. Yet, it is preferable to extract the compound-group from black ginger that has such group in high concentrations. Black ginger refers to the plant academically called “Kaempferia parviflora” that belongs to the genus Kaempferia of the Zingiberaceae family and is spread throughout Southeast Asia.

As a traditional medicine used in Thailand and Laos or the like, such black-ginger extract is used in enhancing vitality, enriching nutrition, lowering blood-sugar levels, revitalizing bodily strength, improving the gastrointestinal tract, preventing vaginal discharge, healing hemorrhoids and preventing hemorrhoidal diseases, nausea, oral ulcers, arthralgia and gastralgia or the like.

The part of black ginger used in obtaining the compound-group is not specifically limited. Yet, it is preferable to use the rhizome of a black ginger that has such compound-group in high concentrations. The type of black ginger is not specifically limited. Any type, whether the rhizome is immature, fully ripen or dried can be used. Preferably, squeezed rhizome should be used, and the type of squeezed rhizome is not specifically limited. Any type can be used, whether the squeezed rhizome is the liquid type or the concentrated dried-powder type.

Yet, special care should be taken in the keeping of either raw rhizome or raw squeezed rhizome. Thus, it is suitable to use sliced and dried rhizome.

When using sliced and dried rhizome, it is preferable to crush the rhizome through an approximately mesh-40 screen in advance by a crusher or the like to extract the rhizome more efficiently.

The extracting-solvent to use and the conditions of temperature or the like is not limited but can be arbitrarily selected and set. As for the solvent, it is possible to use a non-organic solvent such as water solvent, acid solvent, basic solvent or the like as well as an organic solvent such as hydrophilic solvent or acetone solvent or the like. As for a hydrophilic solvent, it is preferable to select one or more lower-alcohol from among methyl alcohol, ethyl alcohol, n-propyl alcohol, isopropyl alcohol or butyl alcohol due to ease of handling and efficient extraction. Yet, it is preferable to extract with a non-organic than with an organic solvent. Especially, it is preferable to use room-temperature water, warm water, hot water or water with a slight amount of acid or ethanol.

At this time, the kind of acid to use is not limited, but it is preferable to use an acetic acid due to safety and good post-handling.

It is preferable to repeat, once or more, the same extraction process on the extracted residue to improve extraction efficiency, in which case the extraction-solvent to use can be of the same kind or of a different kind.

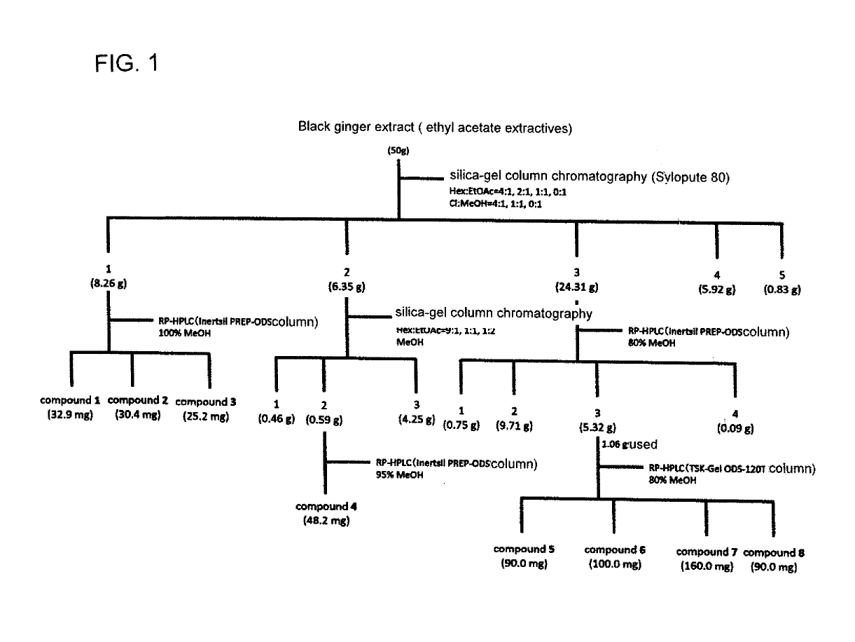

To obtain the compound-group, the above extract is filtered, and the process of centrifugal-separation and fractional distillation is done to remove the insoluble substances and the solvent. Then, the extracted liquid is diluted, concentrated, dried, purified or the like by the usual method to make the energy-metabolic activating agent. The purification method, for example, includes an activated-carbon treatment; a resin-absorption treatment; an ion-exchange resin treatment or a liquid-liquid countercurrent-distribution treatment or the like. Yet, such extract can be used in food or the like without doing the above purification process, since much such extract is not used in food or the like. Specifically, it is possible to obtain a fraction of the compound-group according to the scheme of FIG. 1 of the following example.

Such a fraction of the compound-group can be used, or it can be used after drying it into powder by the spray-drying or freeze-drying method or the like, if needed.

The energy-metabolic activating agent of this invention is characterized in including as an active substance a compound represented by Chemical Formula 1.

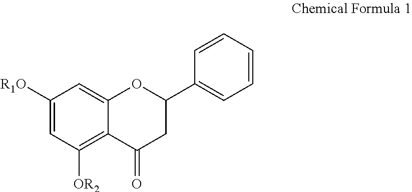

(Of Chemical Formula 1, R1 and R2 respectively mean an alkyl group with hydrogen or with 1˜3-carbon.)

Of the compounds represented in Chemical Formula 1, techtochrysin and 5,7-dimethoxyflavone are preferred.

The method used in obtaining the compounds of Chemical Formula 1 is not limited, but it is preferable to obtain them by extracting them from plants. In obtaining techtochrysin and 5,7-dimethoxyflavone of Chemical Formula 1, it is preferable to use black ginger and to extract and separate them by using the above method.

The energy-metabolic activating agent of this invention can be used as a variety of ingredients (compounds) in different foods and drinks.

Hence, the above expression, “The energy-metabolic activating agent of this invention can be used as a variety of ingredients (compounds) in different foods and drinks” means that different foods can be considered as well as nutritional supplements as specific examples in producing the effects of the energy-metabolic activating agent of this invention. Yet, it does not mean that everyone, including those who do not expect the effects of the energy-metabolic activating agent, can eat such foods.

The blended-percentage showing the effects of the energy-metabolic activating agent is not limited, but the active-substance content in the foods and drinks should be 1 to 20 wt % in total.

The foods and drinks used in mixing the active substance are not limited but include edible oil and fat (salad oil), confectionary (chewing gum, candies, caramels, chocolates, cookies, jellies, gummies, tablet-shaped sweets or other snack food), noodles (Japanese buckwheat noodles called Soba, Japanese wheat noodles called Udon, Chinese noodles called Ramen or the like), dairy food (milk, ice cream, yogurt or the like), seasoning (fermented bean-paste called Miso, soy sauce called Shoyu or the like), soups, drinks (juice, coffee, black tea, green tea, carbonated drinks, sports supplement drinks or the like) and general foods and healthy foods (tablet type, capsule type or the like) and nutritional supplements (nutritious supplement drinks or the like). It is preferable to mix the energy-metabolic activating agents or the like (any one of the above substances (1) to (8) of this invention) with such foods or drinks accordingly.

According to the type of the above foods and drinks, the following ingredients can be added: Glucose, fructose, sucrose, maltose, sorbitol, stevioside, corn syrup, lactose, citric acid, tartaric acid, malic acid, succinic acid, lactic acid, L-ascorbic acid, dl-α-tocopherol, sodium erythorbate, glycerin, propylene glycol, glycerin fatty acid ester, polyglycerol fatty acid ester, sucrose fatty acid ester, sorbitan fatty acid ester, propylene glycol fatty acid ester, Arabian gum, carrageenan, casein, gelatin, pectine, agar-agar (gelatin made from seaweed), vitamin B family, nicotinic-acid amide, pantothenate acid calcium, amino acids, calcium salts, pigment, aroma chemicals, preservatives, or the like.

Also, other antioxidants or compounding ingredients of the energy metabolic activating agent or the like having a health maintenance function include the antioxidant “reduced ascorbic acid” or vitamin C and also the antioxidants, vitamin E, reduced glutacin, tocotrienol, vitamin A derivative, lycopene, rutin, astaxanthin, zeaxanthin, fucoxanthin, uric acid, ubiquinone, coenzyme Q-10, folic acid, garlic extract, allicin, sesamin, lignans, catechin, isoflavone, chalcone, tannins, flavonoids, coumarin, isocoumarines, blueberry extract, ingredients for healthy food (V. (vitamin) A, V.B1, V.B2, V.B6, V.B12, V.C, V.D, V.E, V.P, choline, niacin, pantothenic acid, calcium folic acid, EPA, oligosaccharide, dietary fiber, squalene, soybean lecithin, taurine, dunalliela, protein, octacosanol, egg-yolk lecithin, linoleic acid, lactoferrin, magnesium, chrome, selenium, kalium, hem iron, oyster extract, chitosan, chitin oligosaccharides, collagen, chondroitin, turmeric, sweetroot, extract of Chinese wolfberry fruit called kukoshi, cinnamon, hawthorn (may), ginger, bracket fungus, shijimi clam (corbicula japonica) extract, sweetroot, hawthorn, plantain, chamomilla, chamomile, dandelion, hibiscus, honey, pollen, royal jelly, lime, lavender, rose hip, rosemary, sage, bifidobacteria, streptococcus faecalis, lactobacillus, wheat germ oil, sesame oil, perilla oil, soybean oil, medium chain fatty acid, agaricus, ginko biloba extract, chondroitin, brown rice germ oil, leechee, onion, DHA, EPA, DPA, rubus suavissimus s.lee, plant worm (cordyceps sineusis saccardo), garlic, larvae of a bee, papaya, pu-erh-tea, propolis, Acer nikoense, hericium erinaceum, royal jelly, saw palmetto, hyaluronic acid, collagen, gaba, harp seal oil, shark cartilage, glucosamine, lecithin, phosphatydyl serine, panax notoginseng, mulberry leaf, soybean extract, echinacea purpurea, acanthopanax senticosus, barley extract, olive leaf, olive, gymnema, banaba, salacia reticulata, garcinia, chitosan, saint john's wort, jujube, carrot, passion flower, broccoli, placenta, coix lacryma bobi. Var. ma-yuen, grape seed, peanut skin, bilberry, black cohosh, milk thistle (silybum marianum), laurel, sage, rosemary, apocynum venetum, black vinegar, bitter gourd, maca, carthamus tinctorius (safflower), linseed, oolong tea, flower aculeus, caffeine, capsaicin, xylo-oligosaccharide, glucosamine, buckwheat, citrus, dietary fiber, protein, prune, spirulina, young green barley leaf, nucleic acid, natural yeast, shiitake mushroom (lentinus edodes), Japanese plum, amino acid, extract of deep sea shark, morinda citrifolia, oyster meat, snapping turtle, champinion, common plantain, acerola, pineapple, banana, peach, apricot, melon, strawberry, raspberry, orange, fucoidan, acer nikoense, cranberry, chondroitin sulfate, zinc, iron, ceramide, silk peptide, glycine, niacin, chaste tree, ceramide, L-cysteine, red wine leaf, millet, horsetail, bition, centrlla asiatica, lonicera caerulea, pycnogenol, petasites japonicus, rhubarb, clove, rosemary, catechin, pu-erh, citric acid, beer yeast, mellilot, black ginger, ginger, curcuma zedoaria, nattokinase, ang-khak (Chinese red rice), tocotrienol, lactoferrin, cinnamon, tartary buckwheat, cocoa, citrus junos (yuzu) seed extract, perilla seed extract, litchi seed extract, evening primrose extract, black rive extract, α-lipoic acid, gaba, green coffee bean extract, Japanese butterbur extract, kiwi fruit seed extract, citrus unshiu (Japanese orange—mikan) extract, red ginger extract, astaxanthin, walnut extract, Chinese chive seed extract, red rice extract, cistanche tubulosa (schenk) wight, tremella fuciformis (snow fungus) polysaccharide, fucoxanthin, lingonberry extract, cherry blossom extract, Coprinus comatus extract, rice polyamine, wheat polyamine or the like.

As a specific method in using the energy-metabolic activating agent or the like, it is possible to spray dry or freeze dry such energy-metabolic activating agent or the like together with powdered cellulose to make them into either a powder, a granule, a tablet or a solution, thus making it easier to mix them with foods and drinks. Also, it is possible to dissolve such energy-metabolic activating agent or the like in oil and fat, in ethanol, in glycerin or in a mixture of these substances, thus making a liquid to be able to add such liquid to drinks or solid foods. If necessary, it is also possible to mix the energy-metabolic activating agent or the like in a binder such as Arabian gum or dextrin or the like to make such mixture into a powder or a granule to be able to add such powder or granule to drinks or solid foods.

The energy-metabolic activating agent or the like of this invention can be used as the raw material in medicines (including drugs and quasi-drugs). In the making of drugs, the energy-metabolic activating agent or the like of this invention can be appropriately mixed, for example, with raw materials such as vehicles (glucose, sucrose, white soft-sugar, sodium chloride, starch, calcium carbonate, kaolin, crystalline cellulose, cacao oil, hydrogenated vegetable oil, talc or the like); or as binders (distilled water, normal saline solution, ethanol in water, ethanolic solution, simple syrup, dextrose in water, starch solution, gelatin solution, carboxymethyl cellulose, potassium phosphate, polyvinyl pyrrolidone or the like); or as disintegrating agents (alginate sodium, agar-agar, sodium-hydrogen carbonate, sodium-lauryl sulphate, stearic-acid monoglyceride, starch, lactose, powdered aracia, gelatin, ethanol or the like); or as suppressive agents for disintegration (white soft-sugar, stearin, cacao oil, hydrogenated oil or the like); or as absorption promoters (quaternary-ammonium base, sodium-lauryl sulphate or the like); or as absorbents (glycerin, starch, lactose, kaolin, bentonite, silic acid or the like); or as lubricant agents (purified talc, stearate, polyethyleneglycol or the like).

The energy-metabolic activating agent or the like of this invention can be administered orally in the form of tablets, pills, soft or hard capsules, subtle granules, powders, granules or liquids or the like. However, the energy-metabolic activating agent can also be parenterally administered in different forms such as poultices, lotions, ointments, tinctures or creams or the like.

The applied dosage can be adjusted according to the method of administration or to the condition of the disease or to the age of the patient or the like. Adults can normally take approximately 0.5 to 1,000 mg per day of the active substance, while children can take 0.5 to 500 mg per day.

### Working Examples

This invention is described hereinafter in reference to the examples.

### Working Example: Method for Producing the Black-Ginger Extract and Compound-Group

(1) Method Used in Preparing the Black-Ginger Extract

The black ginger was sliced and dried into 100 kg to obtain the extract. Then, the 100 kg of dried black-ginger was crushed at 80 degrees Celsius for two hours to extract aqueous-ethanol in concentration of 70% ethanol w/w. Then, the ethanol extract was dried, thus getting 3.25 kg of the black-ginger extract. A component analysis by HPLC (high-performance liquid chromatography) of the black ginger showed an amount of 5,7-dimethoxyflavone of 8 wt % or more and a total amount of flavonoid of 35 wt % or more.

(2) Method Used in Producing the Chemical Compound-Group

At 70 degrees Celsius for two hours, 2.0 kg of the crushed black ginger (Kaempferia parviflora) was extracted using 10 kg of 70% ethanol (w/w). The liquid extract was then filtered, and 8 kg of the 70% ethanol (w/w) was added to the residue. Then, another extraction was done in the same way. After that, the above two extracted liquids were mixed together and distilled by a solvent at reduced pressure. Then, as the solid content was 20 to 30%, a double amount of water was added thereto. The water-added extracted liquid was distributed then extracted in ethyl acetate. Each transition at reduced pressure was distilled by a solvent, thus getting the ethyl-acetate transition (of 90.92 g, 4.5%).

The fusible part (50.0 g) of the ethyl acetate obtained was separated according to the purification method as described in FIG. 1.

In other words, five fractions (Fraction 1: 8.26 g, Fraction 2: 6.35 g, Fraction 3: 24.31 g, Fraction 4: 5.92 g and Fraction 5: 0.83 g) were obtained by separating them by normal-phase silica-gel column chromatography (hexane-ethyl acetate: 4:1→2: 1→1:1, v/v→ethyl acetate→chloroform. methanol: 4:1→1:1, v/v→methanol).

Fraction 1 was separated by an HPLC (methanol, Inertsil PREP-ODS), thus getting Compound 1 (5-Hydroxy-3,7-dimethoxyflavone: 32.9 mg), Compound 2 (Techtochrysin: 30.4 mg) and Compound 3 (3,7,4′-Trimethylkaempferol: 25.2 mg). Fraction 2 was separated by a normal-phase silica-gel column chromatography (hexane-ethyl acetate: 9: 1→1: 1→1:2, v/v→methanol), thus getting Fraction 2-1: 0.46 g, Fraction 2-2: 0.59 g and Fraction 2-3: 4.25 g. Fraction 2-2 was separated by an HPLC (methanol-water: 95:5, Inertsil PREP-ODS), thus getting Compound 4 (retusine: 48.2 mg). Fraction 3 was separated by an HPLC (methanol:water=80:20, Inertsil PREP-ODS), thus getting Fraction 3-1: 0.75 g, Fraction 3-2: 9.71 g, Fraction 3-3: 5.32 g and Fraction 4: 0.09 g. A part (1.06 g) of Fraction 3 was separated by an HPLC (ethanol:water=80:20, TSK-Gel ODS-120T), thus getting Compound 5 (Pentamethylquercetin: 90.0 mg, Compound 6 (Trimethylapigenin: 90.0 mg, Compound 7 (Tetramethylkaempferol: 100.0 mg, and Compound 8 (5,7-dimethoxyflavone:160.0 mg). The structures of Compounds 1-8 were identified by a two-dimensional nuclear-magnetic resonator (2D-NMR).

### Test Example 1: Evaluation of the Sugar-Transporter (GLUT4) Gene-Expression Promoting Agent

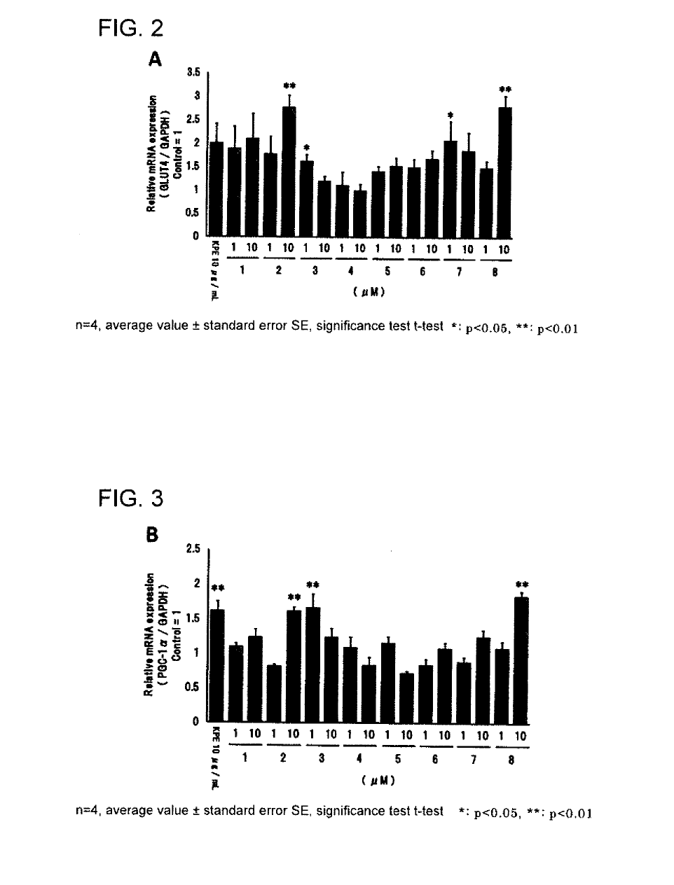

Mouse-muscle myoblast-cell lines C2C12 (cultured in DMEM FCS10%) were seeded in 24 well plates for determining the mRNA expression (1×104 cells/ml) and then were cultured for 24 hours. After 24 hours, the black-ginger extract (10 μg/mL) and the separated fractions (Compounds 1-8) were added to the culture media (DMEM FCS 1%) for differentiation-induction until the concentration became 1 μM or 10 μM (i.e. until the concentration of the sample dissolved in each DMSO (dimethyl sulfoxide) became 0.1% (v/v) regarding the culture media). Then, the black-ginger extract and the separated fractions were cultured for one week. For control, the DMSO was added to the culture media in concentration of 0.1% (v/v). After being cultured for one week, the cells were collected, and the RNA was extracted. Regarding the collected RNA, by using RT-PCR (reverse-transcription polymerase chain-reaction), the expressed mRNA amount of the sugar transporter (GLUT4) was identified. At that time, as an endogenous-control, GAPDH (Glyceraldehyde 3-phosphate dehydrogenase) was used. The result is shown in FIG. 2.

[Result and Effect of the Working Example on Test Example 1]

As shown in FIG. 2, the black-ginger extract (KPE) promotes expression of the sugar-transporter (GLUT4) on the myoblast-cell lines C2C12. On the other hand, eight kinds of compounds from among the KPE were separated and purified. Then, regarding the eight fractions, the expression-promoting effects on the sugar transporter (GLUT4) were evaluated. As a result, the expressed promotion of these separated fractions was identified. Significant expressed-promotion increases were found especially in Compound 2 (Techtochrysin), Compound 3 (3,7,4′-Trimethylkaempferol), Compound 7 (Tetramethylkaempferol) and Compound 8 (5,7-dimethoxyflavone).

### Test Example 2: Evaluation of the PGC-1α-Expression Promoting Effect

Mouse-muscle myoblast-cell lines C2C12 (cultured in DMEM FCS10%) were seeded in 24 well plates for determining the mRNA expression (1×104 cells/ml) and then were cultured for 24 hours. After 24 hours, the black-ginger extract (10 μg/mL) or the separated fractions (Compounds 1-8) were added to the culture media (DMEM FCS 1%) for differentiation induction until the concentration became 1 μM or 10 μM (i.e. until the concentration of the sample dissolved in each DMSO (dimethyl sulfoxide) became 0.1% (v/v) regarding the culture media). Then, the black-ginger extract and the separated fractions were cultured for one week. For control, the DMSO was added to the culture media in concentration of 0.1% (v/v). After being cultured for one week, the cells were collected, and the RNA was extracted. Regarding the collected RNA, by using RT-PCR (reverse-transcription polymerase-chain reaction), the expressed mRNA amount of the PGC-1α was identified. At that time, as an endogenous-control, GAPDH (Glyceraldehyde 3-phosphate dehydrogenase) was used. The result is shown in FIG. 3.

[Result and Effect of the Working Example on Test Example 2]

As shown in FIG. 3, the black-ginger extract (KPE) promotes expression of the PGC-1α on the myoblast-cell lines C2C12. On the other hand, the eight kinds of compounds from among the KPE were separated and purified. Then, regarding the eight fractions, the expression-promoting effects on the PGC-la were evaluated. As a result, the expressed-promotion of the PGC-1α was identified. Significant increases were found, especially in Compound 2 (Techtochrysin) and in Compound 8 (5,7-dimethoxyflavone).

[Effects of the Working Examples]

The above compound-groups proved the increase in the expressions of the sugar transporter (GLUT4) and the PGC-1α (see FIGS. 2 and 3). Regarding such increase in the expressions of the sugar transporter (GLUT4) and the PGC-1α, a structure-activity correlation was identified. Compound-groups having less methoxy in the B-nucleus showed stronger activity, thus showing that the compound-groups described in Chemical Formula 1 have a stronger activity.

In fact, the compound-groups having no methoxy in the B-nucleus showed stronger activity, including Compound 2 (techtochrysin) and Compound 8 (5,7-dimethoxyflavone). Contrarily, the compound-groups having two methoxy groups in the B-nucleus showed lower activity, including Compound 4 (Retsine) and Compound 5 (Pentamethylquercetin) (see FIG. 3). As such, it was identified that the aforementioned compound-groups and the compounds shown by the above Chemical Formula 1 promote the sugar transporter (GLUT4) as a sugar-metabolic transporting factor and promote the expression of the PGC-1α gene that is the factor in energy-metabolic control. Then, it was identified that these compounds have energy-metabolic activating effects.

Therefore, it was confirmed that the aforementioned compound-groups and the compounds shown by the above Chemical Formula 1 can be used as a sugar transporter (GLUT4) gene-expression promoting agent; as a PGC-1α gene-expression promoting agent; and as an energy-metabolic activating agent. It was also confirmed that the black-ginger extract can be used as the PGC-1α gene-expression promoting agent.

The following charts show the blended-percentage of the compounds of the energy-metabolic activating agent. Yet, of this invention, the compounds shown below are not limited to these examples.

### Blending Example 1: Chewing Gums

| | | |
|---|---|---|
| Sugar | 53.0 | wt % |
| Gum base | 20.0 | |
| Glucose | 10.0 | |
| Starch syrup | 16.0 | |
| Aroma chemical | 0.5 | |
| Energy-metabolic | 0.5 | |
| activating agent | | |
| | 100.0 | wt % |

### Blending Example 2: Gummies

| | | |
|---|---|---|
| Reduction sugar | 40.0 | wt % |
| Granulated sugar | 20.0 | |
| Glucose | 20.0 | |
| Gelatin | 4.7 | |
| Water | 9.68 | |
| Kiwi fruit juice | 4.0 | |
| Kiwi fruit flavor | 0.6 | |
| Pigment | 0.02 | |
| Energy-metabolic | 1.0 | |
| activating agent | | |
| | 100.0 | wt % |

### Blending Example 3: Candies

| | | |
|---|---|---|
| Sugar | 50.0 | wt % |
| Starch syrup | 33.0 | |
| Water | 14.4 | |
| Organic acid | 2.0 | |
| Aroma chemical | 0.2 | |
| Energy-metabolic | 0.4 | |
| activating agent | | |
| | 100.0 | wt % |

### Blending Example 4: Yogurt (Hard Type/Soft Type)

| Milk | 41.5 | wt % |
|---|---|---|
| Powdered skim milk | 5.8 | |
| Sugar | 8.0 | |
| Agar-agar | 0.15 | |
| Gelatin | 0.1 | |
| Lactic acid bacterium | 0.005 | |
| Energy-metabolic | 0.4 | |
| activating agent | | |
| Aroma chemical | a minute amount | |
| Water | the rest of the amount | |
| | 100.0 | wt % |

### Blending Example 5: Soft Drinks

| Fructose glucose solution | 30.0 | wt % |
|---|---|---|
| Emulsifying agent | 0.5 | |
| Energy-metabolic activating agent | 0.05 | |
| Aroma chemical | the appropriate amount | |
| Distilled water | the rest of the amount | |
| | 100.0 | wt % |

### Blending Example 6: Soft Capsules

| | | |
|---|---|---|
| Brown rice germ oil | 87.0 | wt % |
| Emulsifying agent | 12.0 | |
| Energy-metabolic | 1.0 | |
| activating agent | | |
| | 100.0 | wt % |

### Blending Example 7: Tablets

| | | |
|---|---|---|
| Lactose | 54.0 | wt % |
| Crystalline Cellulose | 30.0 | |
| A starch-splitting product | 10.0 | |
| Glycerin fatty-acid ester | 5.0 | |
| Energy-metabolic | 1.0 | |
| activating agent | | |
| | 100.0 | wt % |

### Blending Example 8: Granulated Internal Agents (Medicines)

| | | |
|---|---|---|
| Energy-metabolic | 1.0 | wt % |
| activating agent | | |
| Lactose | 30.0 | |
| Cornstarch | 60.0 | |
| Crystalline cellulose | 8.0 | |
| Polyvinyl pvrolidone | 1.0 | |
| | 100.0 | wt % |

### Blending Example 9: Tablet-Shaped Sweets

| | | |
|---|---|---|
| Sugar | 76.4 | wt % |
| Glucose | 19.0 | |
| Glycerin fatty-acid ester | 0.2 | |
| Energy-metabolic | 0.5 | |
| activating agent | | |
| Distilled water | 3.9 | |
| | 100.0 | wt % |

### Blending Example 10: Cat Food

| | | |
|---|---|---|
| Corn | 34.0 | wt % |
| Wheat | 35.0 | |
| Meat meal | 15.0 | |
| Beef fat | 8.9 | |
| Salt | 1.0 | |
| Bonito extract | 4.0 | |
| Energy-metabolic | 1.0 | |
| activating agent | | |
| Taurine | 0.1 | |
| Vitamins | 0.5 | |
| Minerals | 0.5 | |
| | 100.0 | wt % |

### Blending Example 11: Dog Food

| | | |
|---|---|---|
| Corn | 30.0 | wt % |
| Meat (Chicken) | 15.0 | |
| Defatted soybean | 10.0 | |
| Wheat | 25.0 | |
| Chaff and bran | 5.0 | |
| Energy-metabolic | 5.0 | |
| activating agent | | |
| Animal oil and fat | 8.9 | |
| Oligosaccharide | 0.1 | |
| Vitamins | 0.5 | |
| Minerals | 0.5 | |
| | 100.0 | wt % |

### INDUSTRIAL APPLICABILITY

As described above, this invention can provide a safe energy-metabolic activating agent on the muscle cells, with fewer side effects.

1. A sugar transporter (GLUT4) gene-expression promoting agent, including at least one active substance selected from the following: 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone.

2. A sugar transporter (GLUT4) gene-expression promoting agent, including at least one active substance selected from either techtochrysin or 5,7-dimethoxyflavone.

3. A sugar-transporter (GLUT4) gene-expression promoting agent within the muscle cells that includes any one of the chemical compounds as shown in the following Chemical Formula 1. (Of such Chemical Formula 1, R1 and R2, respectively, mean an alkyl group with hydrogen or with 1˜3-carbon.)

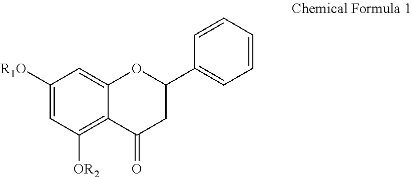

4. A PGC-1α gene-expression promoting agent, including at least one active substance selected from the following: 5-hydroxy-3,7-dimethoxyflavone; techtochrysin; 3,7,4′-trimethylkaempferol; retusine; pentamethylquercetin; trimethylapigenin; tetramethylkaempferol; and 5,7-dimethoxyflavone.

5. A PGC-1α gene-expression promoting agent, including at least one active substance selected from either techtochrysin or 5,7-dimethoxyflavone.

6. A PGC-1α gene-expression promoting agent within the muscle cells, including any one of the chemical compounds as shown in the following Chemical Formula 1. (Of such Chemical Formula 1, R1 and R2 respectively mean an alkyl group with hydrogen or with 1˜3-carbon.)

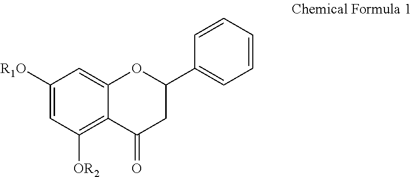

7. An energy-metabolic activating agent of the muscle cells, including the substance described in any one of claims 1 to 6.

8. A sugar-transporter (GLUT4) gene-expression promoting agent, including black-ginger extract as an active substance.

9. A PGC-1α gene-expression promoting agent, including black-ginger extract as an active substance.

10. A composition of food for activating energy metabolism in the muscle cells, including techtochrysin as an active substance.

11. A composition of food for activating energy metabolism in the muscle cells, including 5,7-dimethoxyflavone as an active substance.
